(19) United States
(12) Patent Application Publication
(10) Pub. No.: US 2024/0395396 A1
(43) Pub. Date: Nov. 28, 2024

(54) CAREGIVER WORKLOAD MANAGEMENT

(71) Applicant: Hill-Rom Services, Inc., Batesville, IN (US)

(72) Inventors: Stacie L. Brough, Syracuse, NY (US); David E. Quinn, Moravia, NY (US)

(21) Appl. No.: 18/658,533

(22) Filed: May 8, 2024

Related U.S. Application Data
(60) Provisional application No. 63/503,872, filed on May 23, 2023.

(51) Int. Cl. G16H 40/20; G16H 50/20

(52) U.S. Cl. CPC G16H 40/20; G16H 50/20

(57) ABSTRACT

The present disclosure relates to a caregiver workload management system comprising at least one processing device and a memory device. The memory device stores instructions which, when executed by the at least one processing device, cause the at least one processing device to: receive healthcare facility input data relating to at least one caregiver and at least one patient; analyze the healthcare facility input data to determine at least one patient burden score; determine at least one caregiver burden score using the at least one patient burden score; and issue an alert when the at least one caregiver burden score exceeds a threshold value.

### BACKGROUND

Healthcare facilities deploy caregivers and other staff to care for patients. In certain instances, some caregivers have an unequal patient workload that requires more attention than other caregivers. Unequal patient workloads can cause increased stress for caregivers while decreasing a healthcare facility's efficiency in treating patients.

### SUMMARY

In general terms, the present disclosure relates to managing caregiver workloads. In one possible configuration, a caregiver workload management system receives input data from one or more systems, analyzes the input data, generates burden scores, and issues an alert and/or recommendation when the burden scores exceed a threshold value. Various aspects are described in this disclosure, which include, but are not limited to, the following aspects.

One aspect relates a caregiver workload management system comprising: at least one processing device; and a memory device storing instructions which, when executed by the at least one processing device, cause the at least one processing device to: receive healthcare facility input data associated with at least one caregiver and at least one patient; analyze the healthcare facility input data to determine at least one patient burden score; determine at least one caregiver burden score based on the at least one patient burden score; and issue an alert when the at least one caregiver burden score exceeds a threshold value.

Another aspect relates to a method for method for managing a caregiver workload comprising: receiving healthcare facility input data associated with at least one caregiver and at least one patient; analyzing the healthcare facility input data to determine at least one patient burden score; determining at least one caregiver burden score based on the at least one patient burden score; and issuing an alert when the at least one caregiver burden score exceeds a threshold value.

Another aspect relates to a device for managing caregiver workload in a healthcare facility, the device comprising: at least one processing device; and a memory device storing instructions which, when executed by the at least one processing device, cause the at least one processing device to: receive an overburden alert indicating at least one caregiver burden score exceeds a threshold value, the at least one caregiver burden score associated with at least one caregiver on shift in the healthcare facility; present the caregiver workload alert to the at least one caregiver; and update a workload assigned to the at least one caregiver based on a recommendation included in the overburden alert.

### DESCRIPTION OF THE FIGURES

The following drawing figures, which form a part of this application, are illustrative of the described technology and are not meant to limit the scope of the disclosure in any manner.

FIG. 1 schematically illustrates an example of healthcare facility input data sources and a caregiver workload management system configured to manage the workloads of caregivers within a healthcare facility.

FIG. 2 schematically illustrates an example of a method of generating caregiver workload management outputs that can be performed by the caregiver workload management system of FIG. 1.

FIG. 3 schematically illustrates an example of input data that can be generated by the healthcare facility input data sources of FIG. 1.

FIG. 4 schematically illustrates an example of the caregiver workload management system of FIG. 1.

FIG. 5 schematically illustrates an example of caregiver workload management outputs that are generated by the caregiver workload management system of FIG. 1.

FIG. 6 illustrates an example of a communications device displaying one or more burden scores generated by the caregiver workload management system of FIG. 1.

FIG. 7 illustrates an example of a communications device displaying an overburden alert that can be generated by the caregiver workload management system of FIG. 1.

FIG. 8 illustrates an example of a heat map of a floorplan of a healthcare facility having a plurality of patient burden scores that correspond to a plurality of patients P admitted to the healthcare facility of FIG. 1.

FIG. 9 illustrates an example of an alert displayed by a communications device in the caregiver workload management system of FIG. 1.

FIG. 10 illustrates an example of a method of issuing an overburden recommendation that can be performed by the caregiver workload management system of FIG. 1.

### DETAILED DESCRIPTION

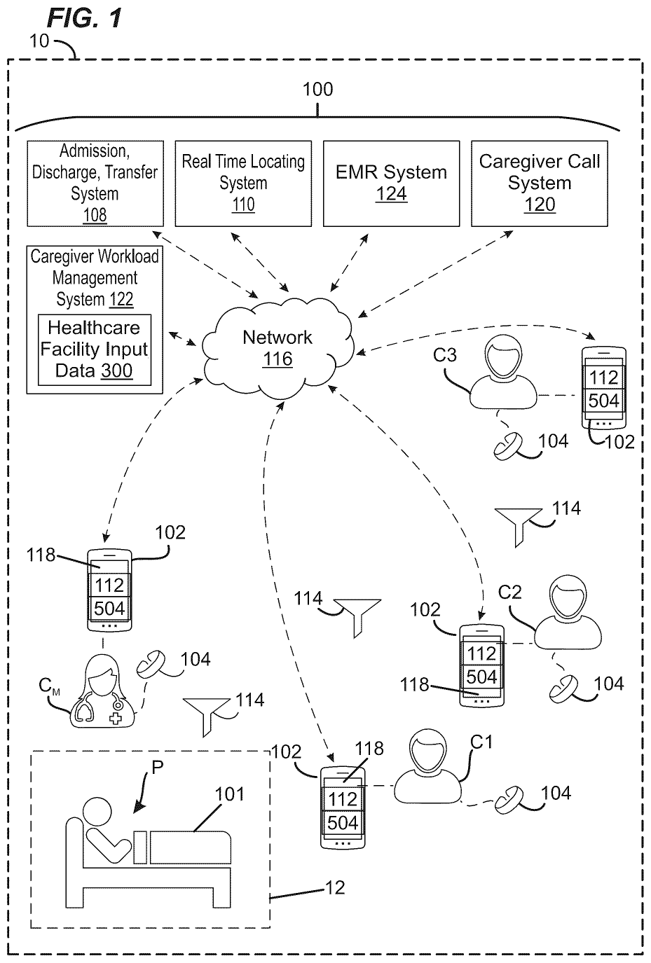

FIG. 1 schematically illustrates a caregiver workload management system 122 configured to manage the workloads of caregivers C within a clinical care environment 10. In certain examples, the clinical care environment 10 is a healthcare facility such as a hospital, a nursing home, a rehabilitation center, a long-term care facility, and the like. As shown in FIG. 1, a patient P is located in a patient environment 12, which can include a room or other designated area within the clinical care environment 10. For example, the patient environment 12 can include a patient room, a department (e.g., emergency department), clinic, ward, or other area within the clinical care environment 10. In this illustrative example, at least one caregiver C is located outside of the patient environment 12. Also, in this illustrative example, at least one caregiver C is located outside of the clinical care environment 10.

Each of the caregivers C operate a communications device 102 on which a caregiver workload management application 112 is installed or is accessible. Examples of the communications devices 102 can include smartphones, tablet computers, or other type of portable computing devices. In further examples, the communications devices 102 can include workstation computers. In some examples, the caregiver workload management application 112 can be downloaded on the communications devices 102. In further examples, the caregiver workload management application 112 can be a web-based or cloud-based application that is accessible on the communications devices 102.

In certain examples, the caregiver workload management system 122 can provide communication channels allowing the caregivers C, administrators, or patients P to leave video, audio, or text messages to discuss outstanding tasks or discuss a patient's condition. In some examples, the caregiver workload management application 112 is an extension of the Voalte® platform available from Hillrom®. In certain examples, the caregiver workload management application 112 incorporates elements of the systems and methods described in U.S. Pat. No. 9,749,825, granted Aug. 29, 2017, titled Connection-Oriented Messaging and Signaling in Mobile Health Networks, and U.S. Pat. No. 9,872,148, granted Jan. 16, 2018, titled Adaptive Mobile Wireless Call Rescue, which are incorporated herein in their entireties.

The caregiver workload management application 112 enables a managing caregiver CM, such as a charge nurse, to assign tasks to other caregivers C. Furthermore, the caregiver workload management application 112 enables the managing caregiver CM to transfer tasks from a first caregiver C1 to a second caregiver C2 when the first caregiver C1 is unable or too busy to perform the tasks such as during a staff shortage at the clinical care environment 10. Staff shortages can occur due to a medical surge when a sudden increase in-patient admissions to the clinical care environment 10 challenges or exceeds the care capacity of the clinical care environment 10. In certain examples, the managing caregiver Cy can transfer tasks from the first caregiver C1 to the second caregiver C2 when the first caregiver C1 has a higher caregiver burden than the second caregiver C2 (i.e., the first caregiver C1 is responsible for a higher number of patients P and/or the patients P that are being cared for by the first caregiver C1 require more attention than the patients P that are being cared for by the second caregiver C2).

The assignment of tasks between the caregivers C is managed by the caregiver workload management system 122 across a communications network 116. As shown in FIG. 1, the communications devices 102 used by the caregivers C are all connected to the communications network 116. The communications network 116 can include any type of wired or wireless connections or any combinations thereof. Examples of wireless connections include Wi-Fi, Bluetooth, and broadband cellular networks including 4G or 5G. In some examples, the communications network 116 includes a broadband cellular network. In some examples the communications network 116 includes the Internet.

In certain examples, a caregiver C can manually request help for certain tasks and patients to lower their burden (i.e., the total amount of attention required by the caregiver C to treat each patient P the caregiver C is responsible for treating). In such examples, a task assignment request from the caregiver C is received by the caregiver workload management system 122 through a connection with the communications device 102 operated by the caregiver C via the communications network 116. The caregiver workload management system 122 can then route the task assignment request to another caretaker C using routing logic based on at least one of an availability of the another caregiver C, a skill level of the another caregiver C, a physical ability of the another caregiver C, one or more preferences of the another caregiver C, and a location of the another caregiver C relative to a location where the task is to be performed in the clinical care environment 10.

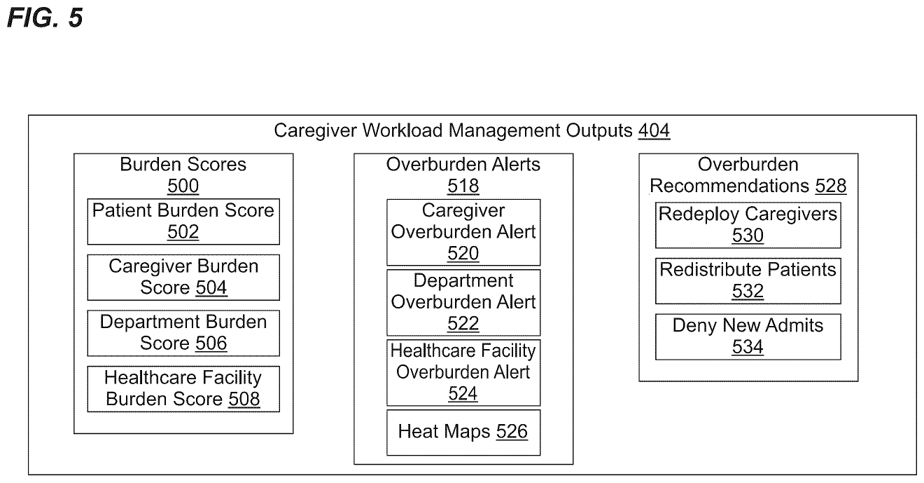

In another example, the caregiver workload management system 122 can proactively assign or triage tasks automatically without requiring manual input from the caregivers C. For example, the caregiver workload management system 122 can determine a staff shortage exists in the clinical care environment 10, and then automatically route task assignment requests to the caregivers C using routing logic based on at least one of availability of the caregivers C, skill level of the caregivers C, physical ability of the caregivers C, preferences of the caregivers C, and locations of the caregivers C relative to the locations where the tasks are to be performed in the clinical care environment 10. In some examples, the caregiver workload management system 122 can proactively assign or triage tasks automatically when a caregiver C is overburdened such that the caregiver C has a caregiver burden score 504 above a threshold value. An example of a caregiver burden score 504 is illustrated and described in further detail with respect to FIG. 5.

As a further illustrative example, the caregiver workload management system 122 can consider the tasks that are accepted and in queue for completion by a caregiver C and can estimate the completion time for each task in the queue based on the type of task, the caregiver C's distance from the patient P, patient P's location as it relates to the task, and other types of factors. The caregiver workload management system 122 can then determine the caregiver C's availability based on the estimated completion time of the tasks accepted by the caregiver C, and can route new task assignment requests to the caregiver C based on their availability. As another example, the caregiver workload management system 122 can optimize the assignment of tasks such as by assigning low-skilled tasks to lower skilled caregivers C to leave open availability of higher skilled caregivers C. Such optimization can especially occur when the caregiver workload management system 122 determines that higher skilled tasks are likely to be assigned based on historical data and trends.

The routing logic that is used by the caregiver workload management system 122 to route the task assignment requests to the caregiver C can be customized. For example, the routing logic can be customized based on the individual needs of the caregiver C, the needs of the team, unit, or department within the clinical care environment 10 where the caregiver C is assigned their shift, or the overall needs of the clinical care environment 10.

Examples of the tasks that can be assigned by the caregiver workload management system 122 to the caregiver C can include, without limitation, assessing a patient's condition, taking vital signs measurements, recording a patients' symptoms and observations, administering medicine to a patient, creating a patient care plan, collaborating with doctors and other caregivers C, and other common types of tasks for providing care in the clinical care environment 10.

The caregiver workload management system 122 can check one or more credentials of the caregiver C to ensure that the tasks are only assigned to caregiver C with appropriate experience, training, and/or physical capabilities. For example, tasks such as retrieving ice chips, feeding a patient, or accompanying a lonely patient may be open to all caregivers C, but a task of helping a patient to the bathroom may only be assigned to caregiver C with basic training.

In some examples, the caregiver workload management system 122 is programmed to perform advance scheduling of tasks. For example, a caregiver C may be scheduled to help feed patients at lunchtime, or if a patient needs a “sitter” to prevent falls, shifts can be scheduled in advance. Administrators may also add tasks that are less time sensitive (e.g., replacing the batteries in clocks) to a queue that could be assigned after more urgent tasks are completed.

Advantages of the caregiver workload management system 122 for the patient P can include improving the overall efficiency of a healthcare facility by evenly distributing tasks among caregivers C, reducing caregiver burnout by providing manageable workloads to each caregiver C, eliminating the need to transfer the patient P to another clinical care environment 10 or location that has care capacity, and minimizing patient deterioration through faster clinical intervention. Further advantages of the caregiver workload management system 122 can include increasing the care capacity of the clinical care environment 10 to provide clinical care for a higher number of admitted patients.

As shown in FIG. 1, one or more healthcare facility input data sources 100 collect healthcare facility input data. For example, an admission, discharge, and transfer (ADT) system 108 is connected to the communications network 116. The ADT system 108 tracks patients from their moment of arrival at the clinical care environment 10 until their departure, and can store relevant patient information such as medical record numbers, names, and contact information. As will be described in more detail below, data acquired from the ADT system 108 can be used to determine whether certain caregivers C are overburdened, which might occur due to a staff shortage in the clinical care environment 10 based on patient volume.

As further shown in the example provided in FIG. 1, the caregivers C each wear or otherwise carry a tag 104 that is detectable by antennas 114 positioned throughout the clinical care environment 10. The antennas 114 are fixed reference points that receive wireless signals from the tags 104. The antennas 114 communicate the wireless signals from the tags 104 to a real-time locating system (RTLS) 110 via the communications network 116. The RTLS 110 uses the data acquired from the antennas 114 to monitor and track the location of the tags 104 (and of the caregivers C) inside the clinical care environment 10.

Alternatively, the location of the caregivers C can be monitored by tracking the movement of the communications devices 102. In examples where the communications devices 102 are portable computing devices such as smartphones or table computers carried by the caregiver C, the location of the communications devices 102 can be tracked by various tracking techniques including multilateration of signals between cell towers of a telecommunications network and the communications devices 102, or by using geo-spatial positioning techniques by satellite navigation systems such as the Global Positioning System (GPS).

In this example, a first caregiver C1 is shown as being physically located in closer proximity to the patient P than second and third caregiver C2, C3. In this example, the routing logic used by the caregiver workload management system 122 can factor in the relative locations of the first, second, and third caregivers C1, C2, C3 with respect to the location where the patient P is located when determining which caregiver C to route a task assignment request to.

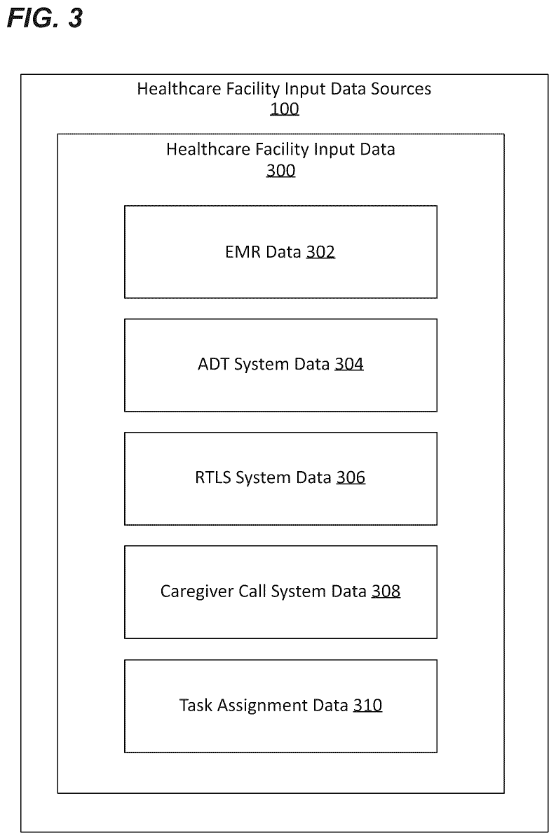

As further shown in the example provided in FIG. 1, an electronic medical records (EMR) system 124, also known as electronic health records (EHR) system, is connected to the communications network 116. The EMR system 124 stores the medical history of the patient P. In certain examples, the EMR system 124 includes EMR data that includes information about a patient's P diagnoses (including a current primary diagnosis), past or current medicines that the patient P is taking, physiological variable measurements of the patient P, clinical interventions provided to the patient P, allergies, immunizations, and treatment plans. The EMR data is illustrated and described in further detail with respect to FIG. 3.

As further shown in the example provided in FIG. 1, a caregiver call system 120 is connected to the communications network 116. The caregiver call system 120 receives requests from the patient P, and can generate and send alerts to a caregiver C to respond to the requests from the patient P. In certain examples, the alert can be routed via the communications network 116 to a communications device 102 used by the caregiver C during their shift. In certain examples, the alert can include a request from the patient P to perform a clinical intervention, such as in response to a triggered alarm. In certain examples, the alert can include information retrieved from the EMR system 124 or from an alarm communication system.

As further shown in the example provided in FIG. 1, a caregiver workload management system 122 is connected to the communications network 116. The caregiver workload management system 122 communicates with the caregiver workload management application 112 over the communications network 116 to determine at least one caregiver burden score 504 and at least one patient burden score 502. The at least one caregiver burden score 504 and the at least one patient burden score 502 can be determined by the caregiver workload management system 122 by analyzing healthcare facility input data 300 collected over the communications network 116 from one or more of the healthcare facility input data sources 100.

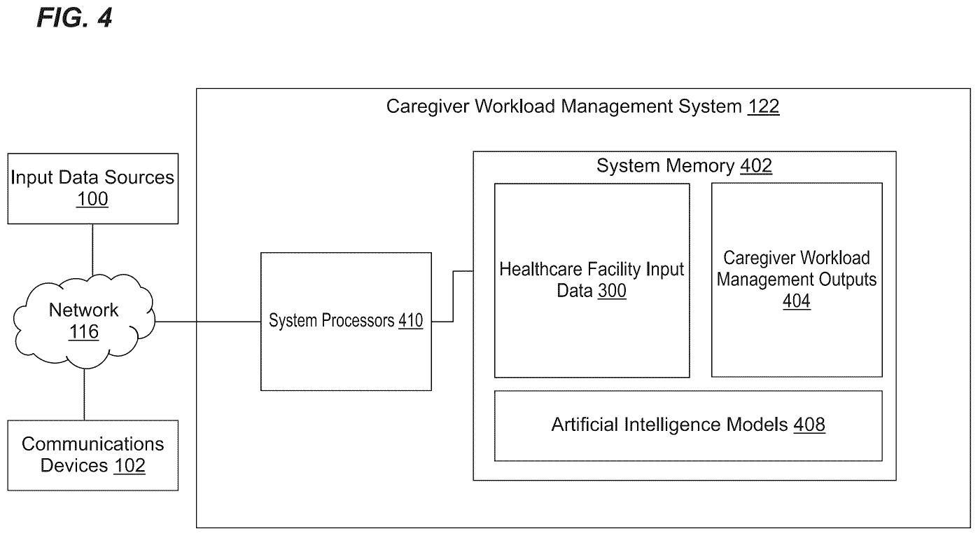

The caregiver burden scores 504 can be used to evaluate the workloads of at least one caregiver C, and tasks can be redistributed between caregivers C to reduce caregiver burnout and increase efficiency within a healthcare facility. The healthcare facility input data 300 is illustrated and described in further detail with respect to FIG. 3. The caregiver workload management system 122 is illustrated and described in further detail with respect to FIG. 4. Examples of alerts and/or recommendations generated by the caregiver workload management system 122 are illustrated and described in further detail with respect to FIGS. 5-9.

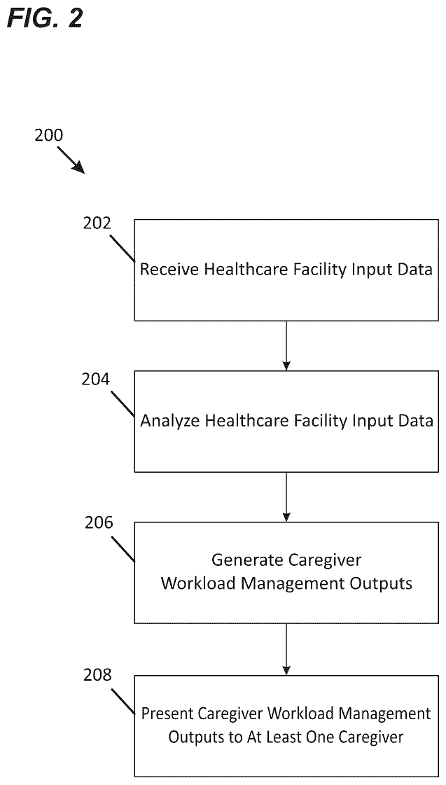

FIG. 2 schematically illustrates an example of a method 200 of generating caregiver workload management outputs. In some examples, the method 200 is performed by the caregiver workload management system 122.

The method 200 includes a step 202 of receiving the healthcare facility input data 300 over the communications network 116 from one or more of the healthcare facility input data sources 100. In certain examples, the one or more healthcare facility systems can include the examples illustrated and described above with reference to FIG. 1, such as the ADT system 108, the RTLS 110, the EMR system 124, and the caregiver call system 120. These systems generate the healthcare facility input data 300, which can be communicated to the caregiver workload management system 122 via the communications network 116. The healthcare facility input data 300 can include one or more inputs relating to caregivers C and patients P, which are described and illustrated in further detail with respect to FIG. 3.

The method 200 further includes a step 204 of analyzing the healthcare facility input data 300. As discussed above with reference to FIG. 1, the caregiver workload management system 122 communicates with the caregiver workload management application 112 over the communications network 116 to determine at least one burden score 500. The at least one burden score 500 can be determined by the caregiver workload management system 122 by analyzing the healthcare facility input data 300. The caregiver burden scores 504 can be used to evaluate the workloads of at least one caregiver C, and tasks can be redistributed between caregivers C to reduce caregiver burnout and increase efficiency within a healthcare facility. Examples of analyzing the healthcare facility input data 300 performed in step 204 are illustrated and described in further detail with respect to FIGS. 3-5.

The method 200 includes a step 206 of generating caregiver workload management outputs and a step 208 of presenting the caregiver workload management outputs to at least one caregiver C. In certain examples, the caregiver workload management outputs 404 include burden scores 500, overburden alerts 518, and overburden recommendations 528. Examples of the caregiver workload management outputs 404, including the burden scores 500, the overburden alerts 518, and the overburden recommendations 528, are described and illustrated in further detail with respect to FIG. 5.

FIG. 3 schematically illustrates examples of healthcare facility input data 300 that can be generated by the healthcare facility input data sources 100 of FIG. 1. As described above, the healthcare facility input data sources 100 can include the ADT system 108, the RTLS 110, the caregiver call system 120, and the EMR system 124. The healthcare facility input data 300 includes EMR data 302, ADT system data 304, RTLS data 306, caregiver call system data 308, and task assignment data 310. The caregiver workload management system 122 analyzes one or more of the EMR data 302, the ADT system data 304, the RTLS data 306, the caregiver call system data 308, and the task assignment data 310, including any combinations thereof, to generate caregiver workload management outputs 404, which are described and illustrated in further detail with respect to FIGS. 5-9.

The EMR data 302 includes information about a patient P's diagnoses, past or current medicines that the patient P is taking, clinical interventions provided to the patient P, vital signs and other physiological variable measurements, allergies, immunizations, and treatment plans. In certain examples, the caregiver C can enter the EMR data 302 into the EMR system 124 using a communications device 102. In certain examples, the EMR data 302 is utilized to generate caregiver burden scores 504 by considering a patient P's condition and estimated clinical interventions that a caregiver C will need to provide to treat the patient's condition.

The ADT system data 304 is generated by the ADT system 108. As described above, the ADT system 108 tracks patients from their moment of arrival at the clinical care environment 10 until their departure, and can store relevant patient information such as medical record numbers, names, and contact information. In certain examples, the ADT system data 304 can be utilized to generate caregiver burden scores 504 by considering the number of patients P that are admitted to a healthcare facility and assigned to a specific caregiver C.

The RTLS data 306 is generated by the RTLS 110. As described and illustrated above with respect to FIG. 1, the RTLS 110 uses the data acquired from the antennas 114 to monitor and track the location of the tags 104 (and the caregivers C) inside the clinical care environment 10. In certain examples, the caregiver workload management system 122 utilizes the RTLS data 306 to generate the patient burden score 502 and the caregiver burden score 504 by considering the time the caregiver C spends with the patient P. In further examples, the caregiver workload management system 122 can utilizes data from one or more cameras positioned inside the patient environment 12 to determine the time the caregiver C spends with the patient P for generating the patient burden score 502 and the caregiver burden score 504.

The caregiver call system data 308 is generated by the caregiver call system 120. As described and illustrated above with respect to FIG. 1, the caregiver call system 120 receives requests from a patient P and sends an alert to a caregiver C to respond to the requested received from the patient P. In certain examples, the caregiver workload management system 122 can analyze the caregiver call system data 308 to calculate a caregiver burden score 504 and a patient burden score 502 by determining a frequency of requests received from a particular patient P, and the type of requests that are received from the particular patient P.

The task assignment data 310 is generated by the caregiver workload management system 122. In certain examples, the task assignment data 310 can be utilized by the caregiver workload management system 122 to calculate a caregiver burden score 504 and a patient burden score 502 by determining a number of tasks assigned to each caregiver and an expected time required to complete each task.

FIG. 4 schematically illustrates an example of the caregiver workload management system 122. The caregiver workload management system 122 receives the healthcare facility input data 300 from one or more of the healthcare facility input data sources 100 over the communications network 116. Examples of the healthcare facility input data sources 100 are illustrated and described in further detail above with respect to FIG. 1. Furthermore, the caregiver workload management system 122 stores the healthcare facility input data 300 within a system memory 402 and processes the healthcare facility input data 300 using a system processor 410. In certain examples, the caregiver workload management system 122 continuously receives the healthcare facility input data 300 from one or more of the healthcare facility input data sources 100, and continuously updates the caregiver workload management outputs 404 over time. In certain examples, the caregiver workload management system 122 provides an explanation for why an update to the caregiver workload management outputs 404 occurred.

The system processor 410 receives and executes instructions from the system memory 402 to process the healthcare facility input data 300 received over the communications network 116. In some examples, this includes utilizing artificial intelligence models 408 to process the healthcare facility input data 300 and generate caregiver workload management outputs 404. The artificial intelligence models 408 can be applied to process the healthcare facility input data 300 to generate the caregiver workload management outputs 404 for certain applications. For instance, a machine learning algorithm can be trained using healthcare facility input data 300 from a large number of patients P. Examples of applications using artificial intelligence are illustrated and described in further detail with respect to FIGS. 5-9.

The system memory 402 includes one or more memories configured to store the healthcare facility input data 300 received via the communications network 116 and the caregiver workload management outputs 404. The system memory 402 can be of various types, including volatile and nonvolatile, removable, and non-removable, and/or persistent media. In some examples, the system memory 402 is an erasable programmable read only memory (EPROM) or flash memory.

In this example, the system memory 402 stores at least one set of healthcare facility input data 300 that includes any combination of the data illustrated and described above with respect to FIG. 3. Examples of the healthcare facility input data sources 100 for recording the healthcare facility input data 300 are illustrated and described above with respect to FIGS. 1-3.

Furthermore, the system memory 402 stores the caregiver workload management outputs 404 that are generated by the system processor 410. In some examples, the system processor 410 utilizes artificial intelligence models 408 to generate the caregiver workload management outputs 404. Examples of caregiver workload management outputs 404 are illustrated and described in further detail with respect to FIGS. 5-9.

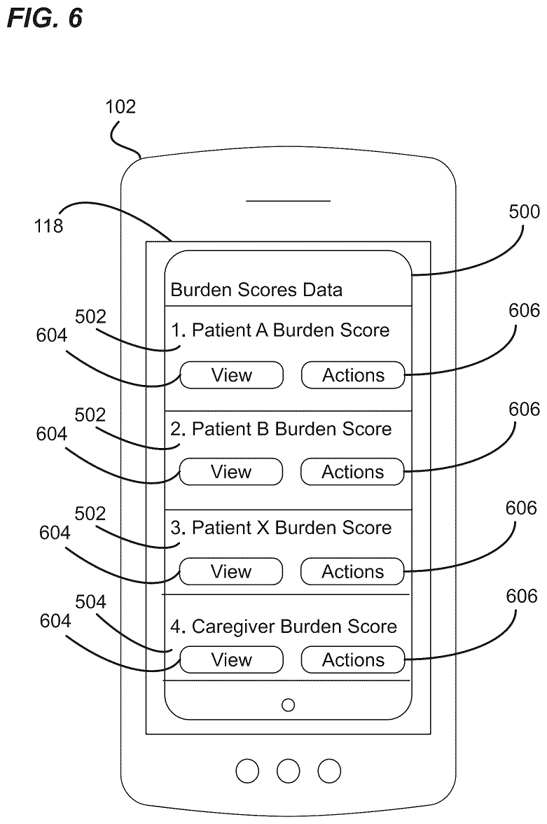

The communications device 102 receives via the communications network 116 data, such as the caregiver workload management outputs 404, from the caregiver workload management system 122. The communications device 102 displays the caregiver workload management outputs 404 for a caregiver C to review. Examples of communications devices 102 are illustrated and described in further detail with respect to FIGS. 6-9.

FIG. 5 schematically illustrates examples of the caregiver workload management outputs 404 that are generated by the caregiver workload management system 122. The caregiver workload management outputs 404 include burden scores 500, overburden alerts 518, and overburden recommendations 528.

The burden scores 500 are calculated by the caregiver workload management system 122. The burden scores are utilized to understand burdens of a healthcare facility at a patient level, a caregiver level, a department level, and a facility level.

The patient burden score 502 represents a burden of each patient P admitted to the healthcare facility. The patient burden score 502 is determined by the caregiver workload management system 122 by considering the healthcare facility input data 300. In certain examples, caregiver workload management system 122 considers at least one of the EMR data 302, the ADT system data 304, the RTLS data 306, and the caregiver call system data 308 in determining the patient burden score 502. In certain examples, the caregiver workload management system 122 considers any combination of one or more of the EMR data 302, the ADT system data 304, the RTLS data 306, the caregiver call system data 308, and the task assignment data 310 for calculating the patient burden score 502.

As an illustrative example, caregiver workload management system 122 can consider the EMR data 302, including a patient's medical history, diagnoses, caregiver orders or instructions to the patient P or other caregivers C regarding the treatment of the patient P (e.g. medication prescriptions, diagnostic tests, treatments, therapies, dietary restrictions, activity limitations, or other medical interventions specific to the patient P), and clinical interventions that have been administered to the patient P, to calculate the patient burden score 502. As another example, the caregiver workload management system 122 considers ADT system data 304, including the patient P's previous admissions to the healthcare facility as well as the time the patient P has currently spent within the healthcare facility, to calculate the patient burden score 502. As another example, the caregiver workload management system 122 considers the RTLS data 306, including the amount of time the caregivers C have spent with the patient P, to calculate the patient burden score 502. As another example, the caregiver workload management system 122 considers the caregiver call system data 308, including the frequency and types of requests received from the patient P, to calculate the patient burden score 502.

In certain examples, the patient burden score 502 is outputted as a numerical value within a range of zero to ten. In certain examples, the patient burden score 502 is outputted as a numerical value within a range of zero to one hundred. In certain examples, the patient burden score 502 is presented to a caregiver C on a communications device 102. In certain examples, the patient burden score 502 is calculated using an algorithm. In certain examples, the patient burden score 502 is calculated using the artificial intelligence models 408.

The caregiver burden score 504 represents a burden workload of each caregiver C within the healthcare facility. The burden workload of each caregiver C is dependent upon factors that can include the number of patients P the caregiver is responsible for providing care to and the patient burden score 502 of each patient P. In certain examples, the caregiver workload management system 122 considers the patient burden score 502, including the factors discussed above, in addition to task assignment data 310 to determine the caregiver burden score 504.

In certain examples, an overburden alert 518 and/or an overburden recommendation 528 can be issued when the caregiver burden score 504 exceeds a predefined threshold. In certain examples, the caregiver burden score 504 is calculated using an algorithm. In certain examples, the caregiver burden score 504 is calculated using the artificial intelligence models 408. In certain examples, the caregiver burden score 504 is outputted as a numerical value within a range of zero to ten or zero to one hundred, or other range of numerical values.

The department burden score 506 and the healthcare facility burden score 508 determine an overall burden of a healthcare department and a healthcare facility, respectively. In some examples, the caregiver workload management system 122 considers the patient burden scores 502 of the patients P admitted to a department within the healthcare facility to calculate the department burden score 506. Alternatively, or additionally, in certain examples, the caregiver workload management system 122 considers the caregiver burden score 504 of each caregiver C on duty within the department to calculate the department burden score 506.

In some examples, the caregiver workload management system 122 considers the patient burden scores 502 of the patients P admitted to the healthcare facility to calculate the healthcare facility burden score 508. Alternatively, or additionally, the caregiver workload management system 122 can consider the caregiver burden score 504 of each caregiver C on shift within the healthcare facility to calculate the healthcare facility burden score 508.

In certain examples, an overburden alert 518 and/or an overburden recommendation 528 is issued when the department burden score 506 or healthcare facility burden score 508 exceed predefined thresholds. In certain examples, the department burden score 506 and the healthcare facility burden score 508 are calculated using an algorithm. In certain examples, the department burden score 506 and the healthcare facility burden score 508 are calculated using the artificial intelligence models 408. In certain examples, the department burden score 506 and the healthcare facility burden score 508 are outputted as numerical values within a range of zero to ten or zero to one hundred, or other range of numerical values.

In certain examples, an average burden score is calculated over a period of time and a current burden score 500 is compared to the average burden score to determine when a caregiver, a department within a healthcare facility, or the healthcare facility is overburdened. In some examples, an overburden alert 518 and/or overburden recommendation 528 is issued when the caregiver workload management system 122 determines the caregiver C, the department within the healthcare facility, or the healthcare facility is overburdened.

The overburden alerts 518 include alerts communicated via the communications devices 102 to indicate that at least one of a caregiver burden score 504, a department burden score 506, and a healthcare facility burden score 508 exceeds a predefined threshold. The overburden alerts 518 can include a caregiver overburden alert 520, a department overburden alert 522, and a healthcare facility overburden alert 524 that correspond to the caregiver burden score 504, the department burden score 506, and the healthcare facility burden score 508, respectively. In certain examples, the overburden alerts 518 include a description of the caregiver C, the department, or the healthcare facility that is overburdened. In certain examples, the overburden alerts 518 are presented to at least one caregiver C (such as a charge nurse) via a communications device 102. In certain examples, the overburden alerts 518 are presented to the at least one caregiver with an overburden recommendation 528 to alleviate the burden of another caregiver C, a department within a healthcare facility, or the healthcare facility overall. In certain examples, the overburden alerts 518 are generated using an algorithm. In certain examples, the overburden alerts 518 are generated using the artificial intelligence models 408.

In certain examples, the overburden alerts 518 include a heat map 526 that illustrates at least one burden score 500. In certain examples, the heat map 526 can include a visual illustration that contrasts areas of a healthcare facility where patients P are admitted with varying patient burden scores 502. In certain examples, the heat map 526 can include a visual illustration that contrasts areas of a healthcare facility that contain caregivers C with varying caregiver burden scores 504. In certain examples, the heat map 526 can include a visual illustration that contrasts different departments within a healthcare facility or different healthcare facilities with varying department burden scores 506 or different healthcare facility burden scores 508. In certain examples, the heat map 526 includes a three-dimensional visualization of the healthcare facility and icons that illustrate areas of the healthcare facility that exhibit high burden scores.

The overburden recommendations 528 include recommendations generated by the caregiver workload management system 122 to reduce at least one of a patient burden score 502, a caregiver burden score 504, a department burden score 506, and a healthcare facility burden score 508. In certain examples, at least one overburden recommendation 528 is provided with at least one overburden alert 518. In certain examples, the overburden recommendations 528 are generated using an algorithm. In certain examples, the overburden recommendations 528 are generated using the artificial intelligence models 408. In certain examples, the overburden recommendations 528 are presented to at least one caregiver C, such as a charge nurse, via a communications device 102.

In certain examples, the overburden recommendations 528 include a recommendation to redeploy caregivers 530. The recommendation to redeploy caregivers 530 can include at least one of removing at least one caregiver C from a healthcare facility staffing record, adding at least one caregiver C to a staffing record, and moving caregivers C to a different department or healthcare facility to treat a different set of patients P. The healthcare facility staffing record includes a record of shifts for each caregiver within the healthcare facility.

In certain examples, the overburden recommendations 528 include a recommendation to redistribute patients 532. The recommendation to redistribute patients 532 can include at least one of adding a patient P to a list of patients assigned to a caregiver C and removing a patient P from the list of patients. In certain examples, patients P can be transferred between caregivers C by viewing an overburden alert 518 and/or an overburden recommendation 528 and providing an action on a communications device 102. Examples of an overburden alert 518 and an overburden recommendation 528 received on a communications device 102 are illustrated and described in further detail with respect to FIGS. 6-9.

In certain examples, the overburden recommendations 528 include a recommendation to deny new patient admissions 534 when a caregiver burden score 504, a department burden score 506, or a healthcare facility burden score 508 exceed a threshold value. It may be desirable to deny new patient admissions in the healthcare facility when the caregivers C are overburdened to encourage patients P to enter another healthcare facility with a surplus of resources.

FIG. 6 illustrates an example of a communications device 102 displaying one or more burden scores 500 generated by the caregiver workload management system 122. In this example, the burden scores 500 include a plurality of patient burden scores 502, including patient burden scores 502 for Patient A, Patient B, or any number of Patients X. Furthermore, the burden scores 500 include a caregiver burden score 504 that displays a caregiver burden score 504 that is calculated using a number of factors that include the number of patients and each patient burden score 502, as illustrated and described above.

In this example, the burden scores 500 are displayed on a display 118 of the communications device 102. In some examples, the display 118 is a touchscreen that both displays outputs and receives tactile inputs from the caregiver C.

Furthermore, in this example, the display 118 includes an icon 604 that is selectable to view additional information, and an icon 606 that is selectable to perform any number of actions relating to the burden score 500. In certain examples, the additional information can include the healthcare facility input data 300 as illustrated and described in further detail with respect to FIG. 3. In certain examples, the actions can include at least one option to request an overburden recommendation 528, to redistribute a patient P to another caregiver, to escalate an overburden alert 518 to another caregiver C (such as a charge nurse), or to perform another action related to caregiver workload management.

Further yet, in certain examples, the actions can provide an option for the caregiver C to accept a task request, decline a task request, or view a caregiver workload management output 404. When the caregiver C selects the option to accept the task request, the caregiver workload management system 122 assigns the task request to the caregiver C for completion. When the caregiver C selects the option to decline the task request, the caregiver workload management system 122 escalates and reassigns the task request to another caregiver C. In certain examples, when the caregiver C selects the option to decline the task request, the caregiver workload management system 122 will provide a recommendation to assign the task to another caregiver C with a lower caregiver burden score 504.

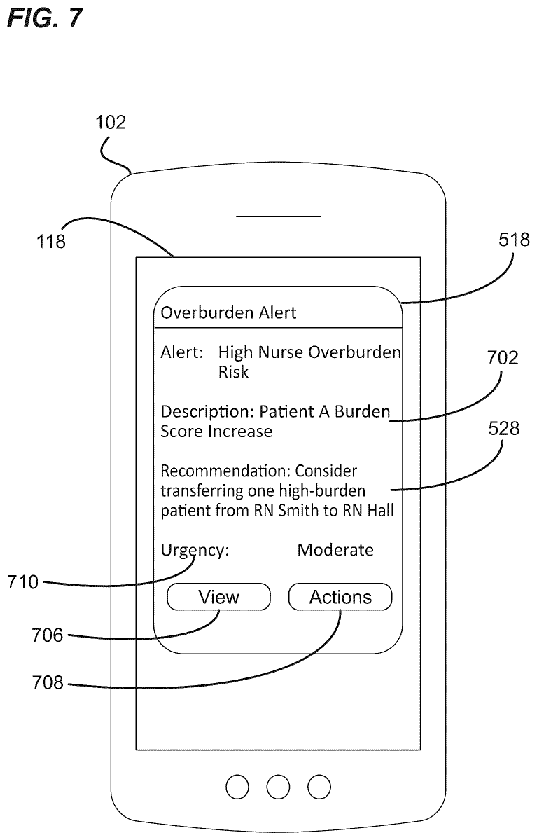

FIG. 7 illustrates an example of a communications device 102 displaying on the display 118 an overburden alert 518 that can be generated by the caregiver workload management system 122. In this example, the overburden alert 518 includes a description 702 of why the caregiver workload management system 122 issued the alert. In the example description, the caregiver workload management system 122 identified that a patient burden score 502 of Patient A increased. In certain examples, a patient burden score 502 may increase due to increased attention that is required from a caregiver C, which may result from changes to a patient's treatment plan, increased calls from the patient P through the caregiver call system 120, or changes to the patient's health condition. Factors that may change a patient burden score 502 are illustrated and described in further detail with respect to FIG. 5.

Further yet, in certain examples, the overburden alert 518 includes an overburden recommendation 528. In this example, the overburden recommendation 528 includes transferring a patient P with a high patient burden score 502 from a caregiver C with a high caregiver burden score 504 to another caregiver C with a lower caregiver burden score 504. Factors that may change a caregiver burden score 504 are illustrated and described in further detail with respect to FIG. 5.

Icons 706, 708 are selectable to view additional information or to perform actions relating to the overburden alert 518, and are substantially similar to the example of the communications device 102 shown in FIG. 6. In certain examples, the icon 706 allows the caregiver C to view any caregiver workload management output 404 or any healthcare facility input data 300. In certain examples, the icon 708 includes options for the caregiver C to escalate the alert to another caregiver C (such as a charge nurse), request a recommendation from the caregiver workload management system 122, or perform any action that would reduce the caregiver burden score 504, such as transferring a patient P to another caregiver as recommended in the overburden recommendation 528 included in the overburden alert 518 shown in this Figure. Furthermore, an urgency description 710 relating to each overburden alert 518 can be included to describe the urgency of the overburden alert 518. In certain examples, the urgency description 710 includes a description of the urgency level that is at least one of a high degree urgency, a moderate degree of urgency, or a low degree of urgency.

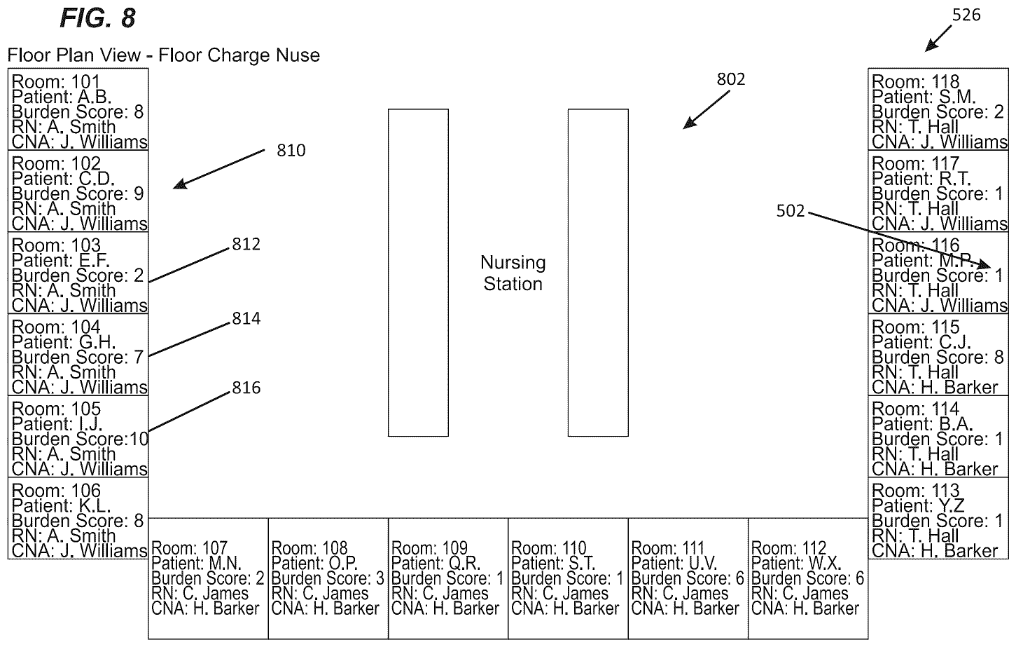

FIG. 8 illustrates an example of a heat map 526 of a floorplan of a healthcare facility having a plurality of patient burden scores 502 that correspond to a plurality of patients P admitted to the healthcare facility. In this example, the heat map 526 includes a plurality of patient descriptors 810. The patient descriptors 810 can include the location of the patient P within the healthcare facility (e.g., a room number), the name of the patient P, the patient burden score 502 corresponding to the patient P, and the caregiver(s) C responsible for providing care to the patient P (e.g., a nurse or a certified nursing assistant).

The patient descriptors 810 can further include visual indicators 812, 814, 816 of the patient burden score 502 corresponding to the patients P shown in the heat map 526. In some examples, the visual indicators 812, 814, 816 correspond to the patients P having a low patient burden score, the patients P having an intermediate patient burden score, or the patients P having a high patient burden score, respectively. In certain examples, the visual indicators 812, 814, 816 can indicate the patient burden scores 502 corresponding to the patient P at the patient location by using coloring and/or shading. In certain examples, darker shading indicates a high patient burden score, whereas a lighter shading indicates a low patient burden score. In certain examples, a red color visually indicates a high patient burden score. In certain examples, a yellow color visually indicates an intermediate patient burden score. In certain examples, a green color visually indicates a low patient burden score.

Furthermore, in this example, the heat map 526 includes other landmarks within the healthcare facility such as a nursing station 802. In the example shown in FIG. 8, the visual indicators 812, 814, 816 show that the left side of the nursing station 802 has a higher burden (i.e., an increased number of red and yellow visual indicators) than the right side of the nursing station 802 (i.e., an increased number of green visual indicators). In this manner, the heat map 526 provides an intuitive display of a distribution of the patient burden scores across the floor plan. This can improve the functioning of the caregiver workload management system 122 because the heat map 526 presents the patient burden scores in a more efficient and intuitive manner. In certain examples, the nursing station 802 includes communications devices 102 such as workstation computers where the caregivers C may exchange messages or receive caregiver workload management outputs 404 (including the heat map 526) from the caregiver workload management system 122.

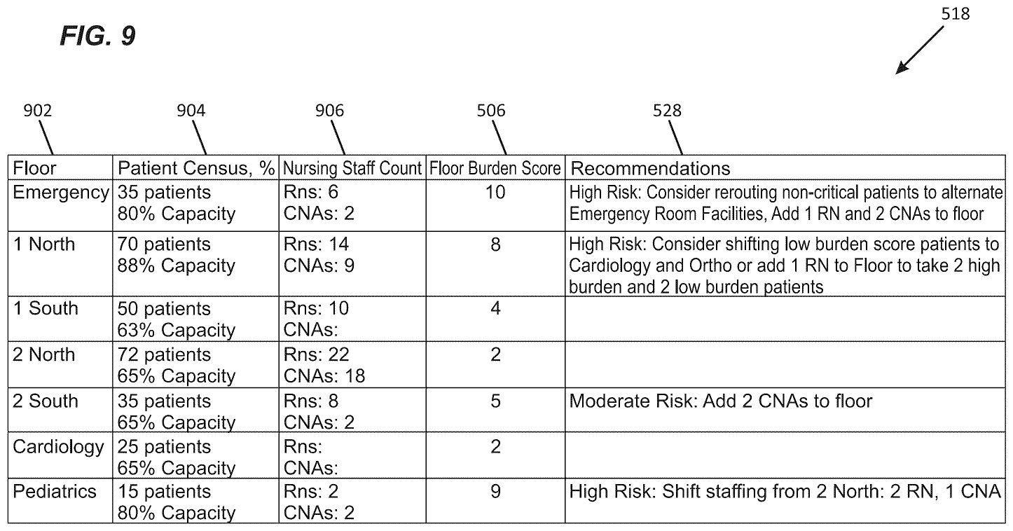

FIG. 9 illustrates an example of an overburden alert 518 that can be displayed by a communications device 102. In this example, the overburden alert 518 includes healthcare facility floor/department information 902, a patient census information 904, a caregiver staff count 906, a department burden score 506, and an overburden recommendation 528 corresponding to the department burden score 506.

The healthcare facility floor/department information 902 identifies a floor or department within the healthcare facility (e.g., 2 South, cardiology, pediatrics, emergency, and the like). Any number of floors and/or departments within a healthcare facility can be identified in the healthcare facility floor/department information 902.

The patient census information 904 includes the number of patients P that are admitted to the floor or department of the healthcare facility identified in the healthcare facility floor/department information 902. The patient census information 904 can also identify a remaining capacity of the floor or department of the healthcare facility. In certain examples, the remaining capacity is displayed as a percentage of admitted patients P relative to a maximum allowed capacity of patients P (e.g., 80% capacity).

The caregiver staff count 906 includes the number of caregivers C that are assigned to each floor or department of the healthcare facility identified in the healthcare facility floor/department information 902. In certain examples, the caregivers C are classified in the caregiver staff count 906 based on education, certifications, degrees of training, and the like. In the example shown in FIG. 9, the caregiver staff count 906 classifies the caregivers C into registered nurses (RNs) and certified nursing assistants (CNAs). Additional categories for classifying the caregivers C are possible.

The department burden scores 506 are each associated with a floor or department of the healthcare facility identified in the healthcare facility floor/department information 902. The department burden scores 506 can include visual indicators such as color coding (e.g., red, yellow, and green shades of color corresponding respectively to high, moderate, and low levels of burden or risk).

The overburden recommendation 528 can include a recommendation to reduce the department burden score 506 such as when the department burden score 506 exceeds a threshold value, or is identified as severe or moderate. In some examples, when the department burden score 506 identifies a light level of burden (e.g., green color coding), an overburden recommendation 528 is not provided. In this example, the overburden recommendation 528 includes a description of the department burden score 506 when the department burden score 506 is indicative of a high risk or moderate risk.

The overburden recommendations 528 can include a recommendation to add additional caregivers C to a floor or department of the healthcare facility to reduce the department burden score 506 calculated for the floor or department of the healthcare facility. In certain examples, the overburden recommendation 528 may assign additional caregivers C to a floor or department of the healthcare facility based on an amount that the department burden score 506 exceeds the threshold value (i.e., healthcare facility floors that greatly exceed the threshold value will receive a greater number of additional caregivers C than healthcare facility floors that minimally exceed the threshold value).

In certain examples, the overburden recommendation 528 can include adding additional caregivers C to a floor or department of the healthcare facility based on a certification, level of experience, and training of the caregivers C. For example, the caregiver workload management system 122 may issue an overburden recommendation 528 to provide additional higher-skilled caregivers (e.g., registered nurses) to a healthcare facility floor that is greatly overburdened with tasks that require a higher degree of training. Conversely, the caregiver workload management system 122 may issue an overburden recommendation 528 to provide additional caregivers who have less training (e.g., certified nursing assistants) to a healthcare facility floor that is greatly overburdened with tasks that require a lower degree of training.

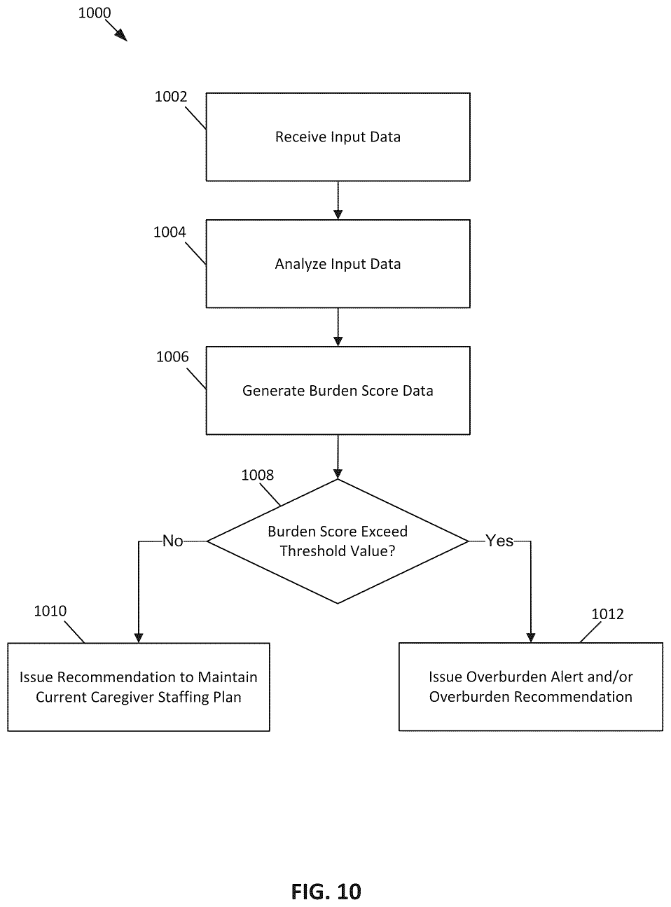

FIG. 10 illustrates an example of a method 1000 of issuing an overburden recommendation. In some examples, the method 1000 is performed by the caregiver workload management system 122. The method 1000 includes a step 1002 of receiving input data from a plurality of systems over the communications network 116. In certain examples, step 1002 includes receiving the healthcare facility input data 300 from at least one of the ADT system 108, the RTLS 110, the EMR system 124, and the caregiver call system 120. The healthcare facility input data 300 and the systems that produce the healthcare facility input data 300 (i.e., the healthcare facility input data sources 100) are described and illustrated in further detail with respect to FIGS. 1-3.

The method 1000 includes a step 1004 of analyzing the input data received in step 1002. In certain examples, step 1002 includes analyzing the healthcare facility input data 300 to produce caregiver workload management outputs 404 as illustrated and described in further detail with respect to FIGS. 3-6. In certain examples, step 1002 includes analyzing the healthcare facility input data 300 using an artificial intelligence model 408 as illustrated and described in further detail with respect to FIGS. 3-6.

The method 1000 includes a step 1006 of generating a burden score 500. The burden scores 500 are described and illustrated in further detail with respect to FIGS. 1-5.

The method 1000 includes a step 1008 of evaluating whether the burden score generated in step 1006 exceeds a threshold value. The comparison between the burden score 500 and the threshold value are described and illustrated in further detail with respect to FIGS. 1-5.

The method 1000 can include a step 1010 of issuing a recommendation to maintain a current caregiver staffing plan when the burden score does not exceed the threshold value (i.e., “No” in step 1008). In some examples, when the burden score 500 does not exceed the threshold value, step 1010 can include issuing an overburden recommendation 528 to consider future changes to caregiver staffing and workloads based on predictions related to the burden scores 500 that are discussed below.

The method can include a step 1012 of issuing an overburden alert 518 and/or overburden recommendation 528 when the burden score exceeds the threshold value. The overburden alert 518 and the overburden recommendation 528 are illustrated and described in further detail with respect to FIGS. 5-9.

In certain examples, the overburden alert 518 and/or overburden recommendation 528 includes a prediction of whether the at least one patient burden score 502 or the at least one caregiver burden score 504 is likely to increase or decrease. In certain examples, the caregiver workload management system 122 evaluates the EMR data 302 to determine whether a patient burden score 502 is likely to increase due to future clinical interventions that may be required to treat the patient's condition. In certain examples, the caregiver workload management system 122 analyzes the ADT system data 304 to evaluate the number of patient's being admitted and/or discharged over time to predict whether a caregiver burden score 504 is likely to increase due to the number of patients P increasing within the healthcare facility or a floor or department within the healthcare facility. In certain examples, the caregiver workload management system 122 analyzes the RTLS data 306 over time to determine whether a caregiver's time spent with a patient is increasing or decreasing. In certain examples, the caregiver workload management system 122 analyzes the caregiver call system data 308 to determine whether the requests from the patient P are increasing over time. In some examples, the caregiver workload management system 122 evaluates the task assignment data 310 to determine whether a caregiver's workload is likely to increase or decrease in the future due to the number of tasks a caregiver C is assigned, a number of resources required to complete each task, and future tasks that are likely to be assigned to the caregiver based on a current patient workload and the EMR data 302.

The block diagrams depicted herein are just examples. There may be many variations to these diagrams described therein without departing from the spirit of the disclosure. For instance, components may be added, deleted, or modified.

The systems and methods described herein provide significant technical advantages. For example, the caregiver workload management system 122 improves the efficiency of healthcare facility task assignment systems by automatically generating alerts to more evenly distribute caregiver workloads within a healthcare facility. Also, the caregiver workload management system 122 is a practical application in healthcare technology that conveys specific alerts and/or recommendations, which may include graphical and/or visual information, in a specific way at a communications device 102 to assist caregivers C. Further yet, the caregiver workload management system 122 analyzes healthcare facility input data to predict future changes to one or more burden scores, and thereby proactively reduce the burden on the caregivers C and the healthcare facilities before a healthcare resources are diminished.

The description and illustration of one or more embodiments provided in this application are not intended to limit or restrict the scope of the invention as claimed in any way. The embodiments, examples, and details provided in this application are considered sufficient to convey possession and enable others to make and use the best mode of claimed invention. The claimed invention should not be construed as being limited to any embodiment, example, or detail provided in this application. Regardless of whether shown and described in combination or separately, the various features (both structural and methodological) are intended to be selectively included or omitted to produce an embodiment with a particular set of features.

Having been provided with the description and illustration of the present application, one skilled in the art may envision variations, modifications, and alternate embodiments falling within the spirit of the broader aspects of the claimed inventions and the general inventive concepts embodied in this application that do not depart from the broader scope.

The various embodiments described above are provided by way of illustration only and should not be construed to be limiting in any way. Various modifications can be made to the embodiments described above without departing from the true spirit and scope of the disclosure.

What is claimed is:

1. A caregiver workload management system comprising:
at least one processing device; and
a memory device storing instructions which, when executed by the at least one processing device, cause the at least one processing device to:
receive healthcare facility input data associated with at least one caregiver and at least one patient;
analyze the healthcare facility input data to determine at least one patient burden score;
determine at least one caregiver burden score based on the at least one patient burden score; and
issue an alert when the at least one caregiver burden score exceeds a threshold value.

2. The system of claim 1, wherein the instructions, when executed by the at least one processing device, further cause the at least one processing device to:
issue a recommendation when the at least one caregiver burden score exceeds the threshold value.

3. The system of claim 2, wherein the recommendation includes a redeployment of the at least one caregiver, or a reassignment of the at least one patient.

4. The system of claim 1, wherein the healthcare facility input data is received from at least one of an electronic medical records (EMR) system, an admission, discharge, and transfer (ADT) system, a real-time locating system (RTLS), and a caregiver call system.

5. The system of claim 1, wherein the instructions, when executed by the at least one processing device, further cause the at least one processing device to:
generate a heat map that illustrates the at least one patient burden score positioned within a floor plan of a healthcare facility.

6. The system of claim 5, wherein the heat map visually distinguishes the at least one patient burden score from other patient burden scores in the floor plan.

7. The system of claim 1, wherein the instructions, when executed by the at least one processing device, further cause the at least one processing device to:
continuously receive the healthcare facility input data; and
update the at least one patient burden score over time.

8. The system of claim 1, wherein the instructions, when executed by the at least one processing device, further cause the at least one processing device to:
predict whether the at least one patient burden score or the at least one caregiver burden score is likely to increase or decrease.

9. The system of claim 1, wherein the instructions, when executed by the at least one processing device, further cause the at least one processing device to:
determine an average caregiver burden score from a plurality of caregiver burden scores calculated for a healthcare facility; and
compare the at least one caregiver burden score to the average caregiver burden score.

10. The system of claim 1, wherein the healthcare facility input data is analyzed using an artificial intelligence model.

11. The system of claim 10, wherein the instructions, when executed by the at least one processing device, further cause the at least one processing device to:
train the artificial intelligence model by receiving an input from a caregiver to validate or reject a recommendation based on the alert.

12. The system of claim 1, wherein the instructions, when executed by the at least one processing device, further cause the at least one processing device to:
issue a recommendation to reassign the at least one patient when the at least one caregiver burden score exceeds a second caregiver burden score by a certain threshold.

13. A method for method for managing a caregiver workload comprising:
receiving healthcare facility input data associated with at least one caregiver and at least one patient;
analyzing the healthcare facility input data to determine at least one patient burden score;
determining at least one caregiver burden score based on the at least one patient burden score; and
issuing an alert when the at least one caregiver burden score exceeds a threshold value.

14. The method of claim 13, further comprising:
issuing a recommendation when the at least one caregiver burden score exceeds the threshold value, wherein the recommendation includes at least one of a redeployment of the at least one caregiver and reassignment of the at least one patient.

15. The method of claim 13, wherein the healthcare facility input data includes at least one of electronic medical records (EMR) data, admission, discharge, and transfer (ADT) data, real-time locating system (RTLS) data, caregiver call system data, and staff assignment data.

16. The method of claim 13, further comprising:
generating a heat map that illustrates the at least one patient burden score that corresponds to the at least one patient positioned within a healthcare facility floor plan.

17. The method of claim 13, further comprising:
predicting whether the at least one patient burden score or the at least one caregiver burden score is likely to increase or decrease.

18. The method of claim 13, further comprising:
issuing a recommendation to redistribute the at least one patient when the at least one caregiver burden score exceeds a second caregiver burden score by a certain threshold.

19. A device for managing caregiver workload in a healthcare facility, the device comprising:
at least one processing device; and
a memory device storing instructions which, when executed by the at least one processing device, cause the at least one processing device to:
receive an overburden alert indicating at least one caregiver burden score exceeds a threshold value, the at least one caregiver burden score associated with at least one caregiver on shift in the healthcare facility;
present the caregiver workload alert to the at least one caregiver; and
update a workload assigned to the at least one caregiver based on a recommendation included in the overburden alert.

20. The device of claim 19, wherein the at least one caregiver burden score is calculated based on patient burden scores of patients assigned to the at least one caregiver.
